(19) United States
(12) Patent Application Publication
(10) Pub. No.: US 2020/0251195 A1
(43) Pub. Date: Aug. 6, 2020

(54) PATIENT ASSESSMENT SYSTEM

(71) Applicants: Matti Palo, Covington, LA (US); Benjamin Legum, Bluemont, VA (US)

(72) Inventors: Matti Palo, Covington, LA (US); Benjamin Legum, Bluemont, VA (US)

(73) Assignee: ORTHOBISTRO LLC, Covington, LA (US)

(21) Appl. No.: 16/265,304

(22) Filed: Feb. 1, 2019

(51) Int. Cl. G16H 10/60; G16H 15/00; G06F 16/22

(52) U.S. Cl. CPC G16H 10/60; G06F 16/22; G16H 15/00

(57) ABSTRACT

A patient assessment system having a computing system including a processor configured to provide a plurality of routines to a patient using an avatar on a display is provided. A sensor array is configured to capture patient data and transmit patient data to a memory in operable communication with the processor. The memory is configured to store a plurality of patient data received from the sensor array. An artificial intelligence engine is in communication with the memory to receive and analyze patient data for one or more abnormalities. A patient report generator is in communication with the artificial intelligence engine and to provide a patient reports to an interface configured to display reports to the user.

### TECHNICAL FIELD

The embodiments presented provide a patient assessment system configured to measure, store, and analyze a variety of data corresponding to a patient.

### BACKGROUND

One frequently encountered challenge by medical professionals was to evaluate a patient's wellbeing at a targeted area. Current devices are limited to non-standardized collection of data or visually inspecting for inconsistencies and abnormalities. These processes are time-consuming, expensive, and inconsistent.

Properly examining a patient requires a significant amount of analysis to be performed in a very short period of time. Often, once an initial observation and initial diagnosis are made, a referral can be made to a specialist for further analysis and screening. This can result in inconsistencies and disconnect between the patient and their medical data.

The technical resources that go into the delivery of healthcare have been studied extensively. Advances are frequent in the area of diagnostic testing, therapeutics, and pharmaceuticals. While these areas are in constant flux, a need still exists to maximize efficiency within the practice. Since, medical professionals often only spend a few minutes with each patient, an ever-present need exists to maximize the efficiency, accuracy, and efficacy of this encounter. The ability of the medical specialist to identify the musculoskeletal problem directly impacts the success of any particular treatment protocol.

Current screening techniques include manual history taking and physical exams, X-Rays, MRI, and CT scans of the patient. These methods have become widely adopted into the practice of treating joint problems and addressing joint performance issues. However, each of these methods can lead to inaccurate and inconsistent patient data, lengthening the time to recovery. Further, these methods often require multiple specialists and appointments for the problem to be correctly assessed.

Strategies must be developed to enhance the quality of care with the amount of time available.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

Embodiments herein provide for a patient assessment system having a computing system, including a processor, configured to provide a plurality of routines to a patient using an avatar. A sensor array is configured to capture patient data and transmit patient data to a memory in operable communication with the processor. The memory is configured to store a plurality of patient data received from the sensor array. An artificial intelligence engine is in communication with the memory to receive and analyze patient data for one or more abnormalities. A patient report generator is in communication with the artificial intelligence engine and to provide patient reports to an interface configured to display reports to the user.

In one aspect, a comparator is configured to compare at least one data set stored in the memory to determine a difference between two or more values in the at least one data set. A diagnosis tool can be used to determine if the difference is above a threshold value. The threshold value is used to elucidate a diagnosis and transmit the diagnosis to at least one of the plurality of reports.

In one aspect, the plurality of reports is stored in the memory permitting a comparing module to compare the plurality of reports for each patient over time.

In one aspect, the user selects at least one of the plurality of routines. The sensor array transmits the patient data in real-time to the artificial intelligence engine in real-time and may change the routine in view of the real-time patient data.

In one aspect, the sensor array is disposed within a dimensioned base unit including an upper assembly and a lower assembly. The sensor array includes at least one of the following: at least one infrared sensor, at least one photographic device, and at least one dynamometer. The sensor array is configured to transmit at least, but not limited to, one of the following: Range of motion data, electrical impedance data, volumetric data, thermographic data, strength data, and optical imaging data.

In one aspect, the plurality of reports is stored in the memory and a comparing module compares the plurality of reports.

In one aspect, the patient assessment system includes an avatar performing the plurality of routines on the display.

Moreover, in accordance with the embodiments, other aspects, advantages, and novel features of the present embodiments will become apparent from the following detailed description in conjunction with the drawings.

### BRIEF DESCRIPTION OF THE DRAWINGS

A more complete understanding of the present invention and the advantages and features thereof will be more readily understood by reference to the following detailed description when considered in conjunction with the accompanying drawings wherein:

FIG. 1 illustrates a perspective view of a patient interacting with the patient assessment apparatus, according to some embodiments;

FIG. 2 illustrates a perspective view of the patient assessment apparatus, according to some embodiments;

FIG. 3 illustrates an exploded view of the lower assembly, according to some embodiments;

FIG. 4 illustrates a perspective view of the lower assembly and sensors, according to some embodiments;

FIG. 5 illustrates an exploded view of the lower assembly and upper surface, according to some embodiments;

FIG. 6 illustrates a top plan view and a perspective view of the lower assembly, according to some embodiments;

FIG. 7 illustrates a schematic of the patient assessment system configuration, according to some embodiments;

FIG. 8 illustrates a perspective view of a patient interacting with the patient assessment apparatus, according to some embodiments;

FIG. 9 illustrates a cutaway view of the dynamometer, according to some embodiments,

FIG. 10 illustrates a block diagram of the patient assessment system, according to some embodiments;

FIG. 11 illustrates a patient results interface, according to some embodiments;

FIG. 12 illustrates a patient search interface, according to some embodiments;

FIG. 13 illustrates a patient exam interface, according to some embodiments;

FIG. 14 illustrates a patient report interface wherein the user is selecting a patient from the database, according to some embodiments;

FIG. 15 illustrates a report interface for the left wrist of the patient, according to some embodiments;

FIG. 16 illustrates a body measurement report interface, according to some embodiments;

FIG. 17 illustrates a range-of-motion report interface, according to some embodiments;

FIG. 18 illustrates a volumetric analysis interface, according to some embodiments;

FIG. 19 illustrates a photographic report interface, according to some embodiments;

FIG. 20 illustrates a thermographic report interface, according to some embodiments;

FIG. 21 illustrates a strength measurement interface, according to some embodiments; and

FIG. 22 illustrates a block diagram of the artificial intelligence engine, according to some embodiments.

### DETAILED DESCRIPTION

The specific details of the single embodiment or variety of embodiments described herein are to the described apparatus. Any specific details of the embodiments are used for demonstration purposes only and not unnecessary limitations or inferences are to be understood therefrom.

Before describing in detail exemplary embodiments, it is noted that the embodiments reside primarily in combinations of components related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first” and “second” and the like, may be used solely to distinguish one entity or element from another entity or element without necessarily requiring or implying any physical or logical relationship or order between such entities or elements.

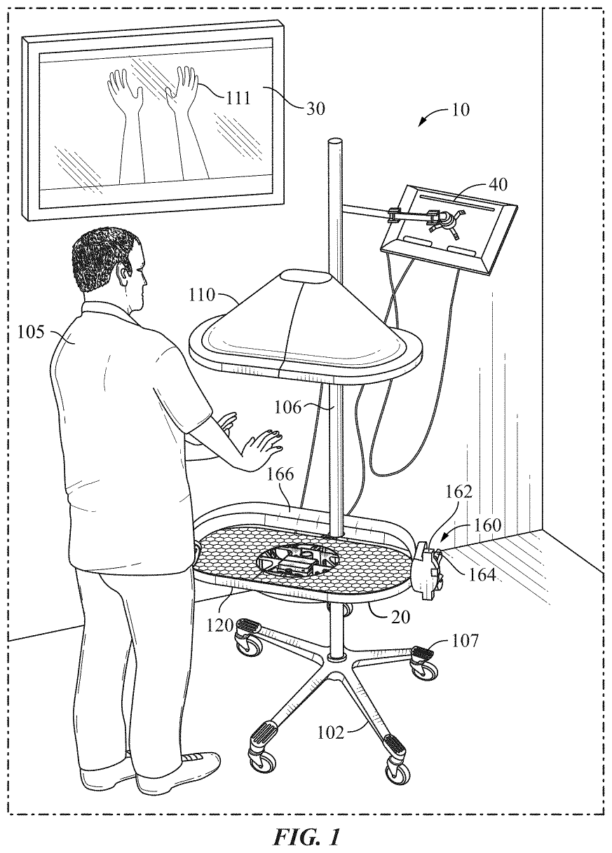

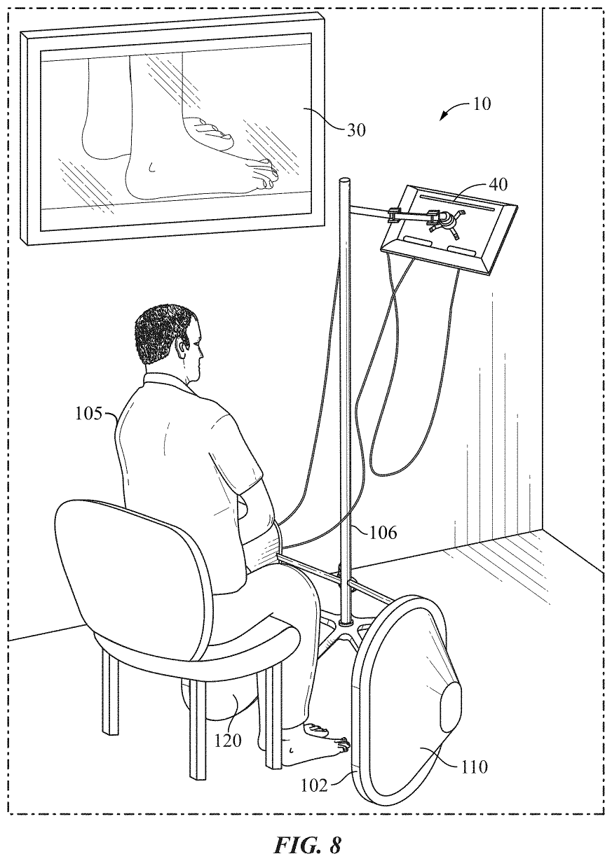

The embodiments presented herein provide a patient assessment apparatus 10 having a sensor array 25 disposed within a base unit 20 dimensioned to fit around the patient's 105 hands and wrist, as shown in FIG. 1. In some embodiments, the base unit 20 is positioned such that the apparatus measures the foot, ankle, knee, and leg of the patient 105 (see FIG. 8). The interior of the base unit 20 includes the sensor array 25 which may move throughout the base unit 20 to collect data corresponding to a patient's movement, a selected area of the body, temperature, power output (i.e., strength), and aesthetic appearance. Display 30 shows an avatar 111 having a routine to the patient.

While the present embodiment provides for a base unit 20 configured to scan the hand, fingers, wrist, and forearm of the user, the base unit can be configured in a variety of dimensions such that alternate areas and specific joints can be scanned.

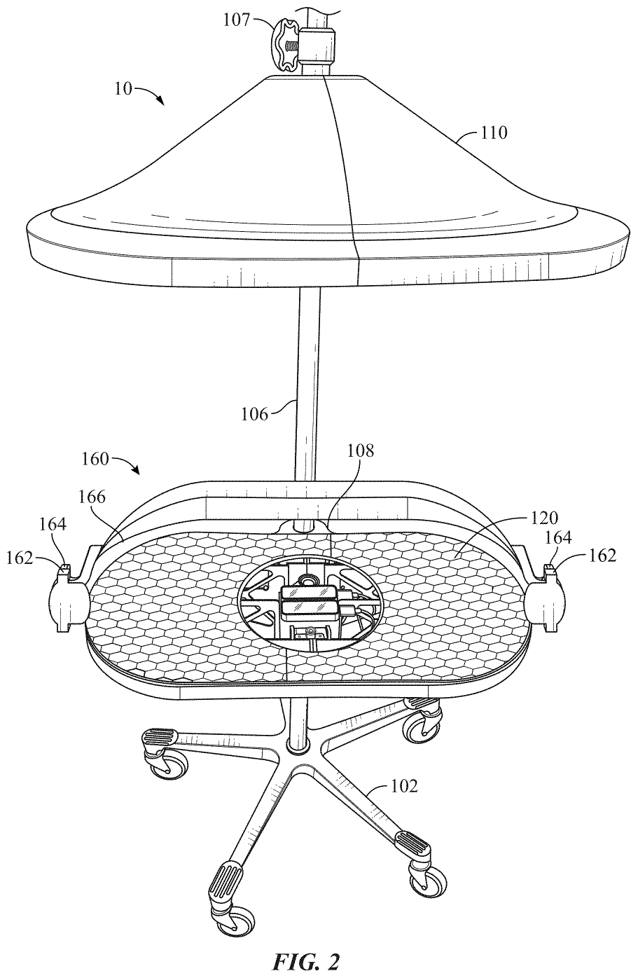

In reference to FIG. 1 and FIG. 2, the apparatus 10 is illustrated comprising an upper assembly 110 and a lower assembly 120. Each of the upper and lower assemblies 110,120 are constructed, assembled, and function uniformly. For simplicity, the lower assembly 120 is discussed in detail throughout with the understanding that the upper assembly 110 is positioned to vertically mirror the lower assembly 120 as shown in FIGS. 1 and 2. An aspect of some embodiments are directed to an apparatus 10 mounted to a base unit 20 constructed to support the upper assembly 110 and lower assembly 120. The base assembly 102 rests movably on the floor or similar surface. In at least one embodiment, the base assembly 102 includes a plurality of wheels allowing the apparatus 10 to be quickly moved. Each wheel can include a locking mechanism 107 to ensure the stability of the apparatus 10 during an interaction with the patient 105.

The lower assembly 120 is positioned at a first height, and the upper assembly 110 is positioned at a second height along the length of the member 106. In one aspect, the height of first and second heights are adjustable and rotatable by one or more adjustment mechanisms 109 dependent on patient 105 height or anatomy of interest. In the illustrated embodiment, member 106 is configured as an elongated cylindrical member to support the weight of the lower and upper assemblies 120, 110. Each of the upper and lower assemblies 110, 120 are movably coupled to the member 106 via pivoting members 108 configured as an aperture to permit the member 106 to pass therethrough. Pivoting member 108 can permit each of the lower assembly and upper assembly 110, 120 to optionally rotate around an axis relative to the base unit 20. Preferentially, each assembly 110, 120 rotates around the member 106. Rotation can be optionally powered by an actuator placed between the member 106 and each assembly 110, 120. Alternate embodiments permit manual rotation of each assembly 110, 120 which can each be locked into the desired position.

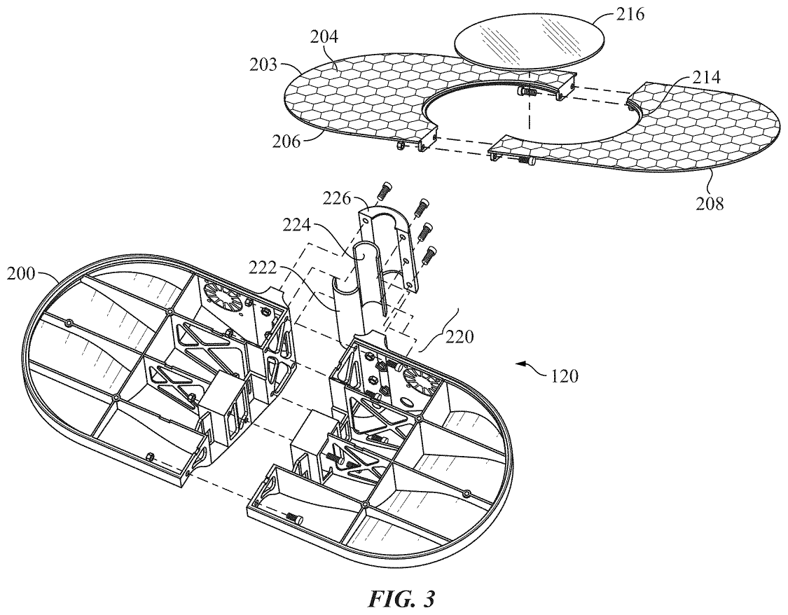

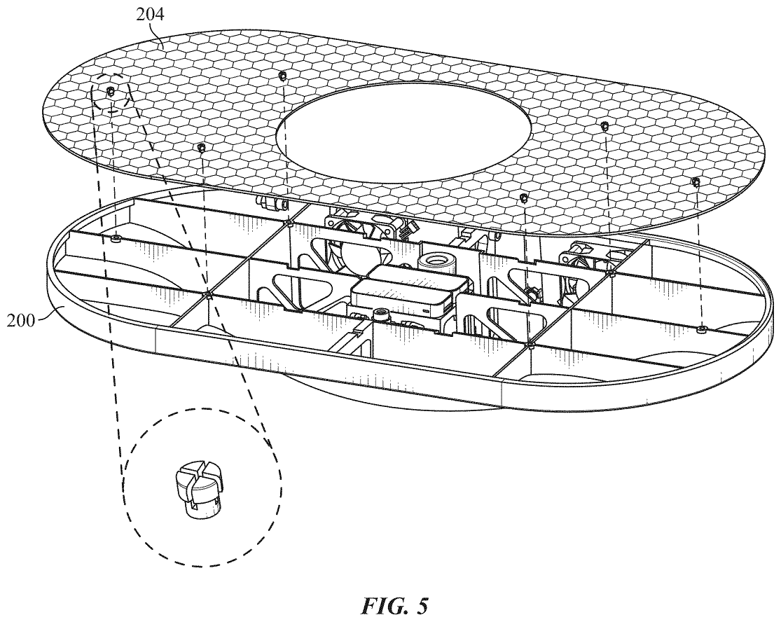

FIG. 3 illustrates an exploded view of the lower assembly 120 comprised of a housing 200 dimensioned to contain the plurality of sensors in an array 25 (see FIG. 1). The lower assembly 110 includes a releasably engaged upper surface 204 to fasten to the housing 200. In one embodiment, the housing edge 201 and upper surface edge 203 are similarly dimensioned such that one component is received by the other (see FIG. 5). The upper surface 204 is configured having a left side 206 and right side 208 which are fastened to one another at connection points at a medial axis. The aperture 214 is dimensioned to receive a transparent, or partially transparent cover 216 which permits light to pass therethrough to the sensors within the lower assembly 110. The transparent cover 216 can be constructed of a suitable transparent or semi-transparent material including polymethyl methacrylate (“PMMA”) or similar thermoplastic utilized when tensile strength, flexural strength, transparency, polishability, and UV tolerance are more important than impact strength, chemical resistance, and heat resistance.

A mounting assembly 220 can fasten to the proximal side 221 (shown in FIG. 2) (in relation to the member 106). The mounting assembly can include inner sleeve components 222, 224 to fittingly engage the member 106. A mounting assembly housing 226 retains the inner sleeve components 222, 224 therein. Each sleeve 222, 224 provides a friction fit to member 106 retaining each of the upper assembly 110 and lower assembly 120 at a first and second height respectively.

The housing 200 can be constructed as a truss having cross members spanning both the length and width of the housing 200 to intersect one another. The truss cross members provide an anchor point for each sensor of the sensor array 25 or another interior component in the lower assembly 120.

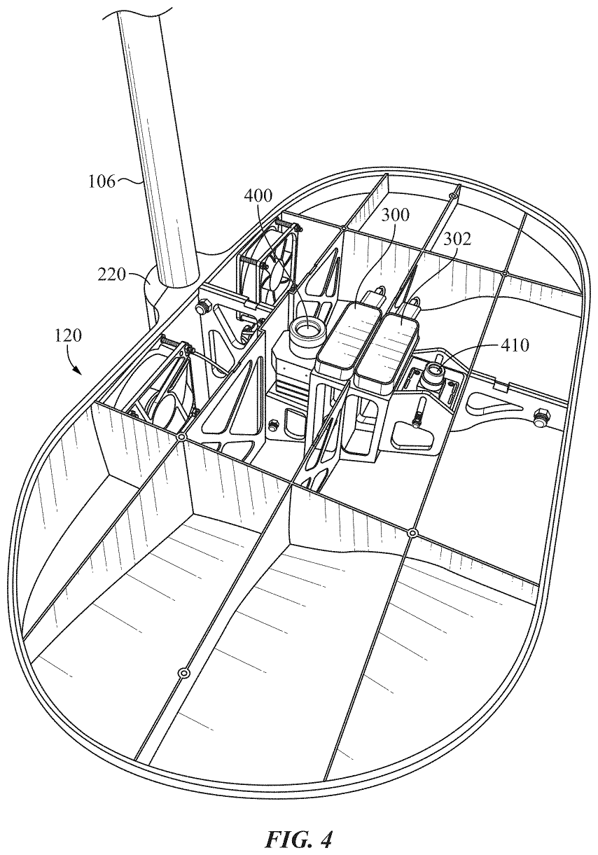

As mentioned, the apparatus 10 includes a sensor array 25 as illustrated in FIGS. 4-6. Each sensor is configured to gather data in relation to the patient 105 and output the data for analysis. In one aspect, the sensor array 25 includes two or more motion sensors 300, 302 for the collection and analysis of data related to the motion and angles of the patient's anatomy. During use, a patient places his or her hands within the range of the motion sensor(s) 300, 302 and moves his arm, wrist, hand, or finger. The system 10 may be provided with an avatar 111 (see FIGS. 1 and 8) to allow the patient 105 to interact with a virtual routine, thus providing an impetus for movement. In one embodiment, the motion sensors 300, 302 include the infrared camera(s) configured to sense infrared Light Emitting Diodes (LED's). Each infrared camera is used to track infrared light outside the visible light spectrum. Each camera has a wide-angle lens to permit a broad interaction space.

In some embodiments, an optical camera is used to calculate the range of motion of the patient. The optical camera senses the anatomy of the patient and utilizes motion and angles between anatomical points to determine the range of motion of the patient.

The embodiments illustrated may or may not require the patient to interact with virtual objects. However, the use thereof is contemplated. In any interaction, data is gathered related to the patient's 105 motion or position and transmitted to a computer system for further processing. The motion sensors 300,302 are utilized to the patient's 105 hands as they perform a range-of-motion routine. The sensor array 25 optically measures the joint and body positioning. The motion sensors 300, 302, and the optical images or infrared data thereof can be utilized to calculate volumetric data of the hands and fingers.

In a preferred embodiment, a pair of motion sensors 300, 302 adhere to a surface of the housing 200 within each of the upper and lower assemblies 110, 120. Each motion sensor 300, 302 is positioned such that the cameras and infrared LED's can collect and emit light through the transparent cover 216 to collect data from the patient 105. A first motion sensor 300 can be configured to track the motion of the patient 105, while the second motion sensor 302 is configured for interaction with an avatar 111.

In one embodiment, the system 10 includes a pair of light sources which can be disposed to either side of the motion sensors 300, 302. Light sources can be infrared light sources of generally conventional design, e.g., infrared light-emitting diodes (LEDs), and cameras can be sensitive to infrared light. A set of filters can be placed in front of the cameras to filter out visible light so that only infrared light is registered in the images captured by cameras. In some embodiments where the object of interest is a person's hand or body, use of infrared light can allow a motion-capture system to operate under a broad range of lighting conditions and can avoid various inconveniences or distractions that may be associated with directing visible light into the region where the person is moving.

For example, lasers or other light sources can be used instead of LEDs. For laser setups, additional optics (e.g., a lens or diffuser) may be employed to widen the laser beam (and make its field of view similar to that of the cameras). Useful arrangements can also include short and wide-angle illuminators for different ranges. Light sources are typically diffuse rather than specular point sources; for example, packaged LEDs with light-spreading encapsulation are suitable.

In a preferred embodiment, and in further reference to FIG. 4 the sensor array 25 includes image sensors 400 and 410. A variety of imagery can be captured, however, in the present embodiment, a thermal imaging device and high definition photography device are used to capture data related to the patient 105. This device can be attached within both the upper and lower assemblies 110, 120. This data can be used to assess heat, swelling and other joint-related concerns. Photography is used to capture images of the patient 105 and photogrammetry is utilized to analyze the image and calculate sizes of wounds, scars, lesions, and other formations. In a preferred embodiment, each camera is positioned on either side of the motion sensors 300, 302.

In alternate embodiments, each camera can be any type of camera, including visible-light cameras, infrared (IR) cameras, ultraviolet cameras or any other devices (or combination of devices) that are capable of capturing an image of an object and representing that image in the form of digital data. Data from the IR camera can be used to calculate and generate a volumetric analysis for specific areas of muscle atrophy and/or hypertrophy. Additionally, the IR camera may be used in conjunction with data gathered from a thermographic camera. The particular capabilities of the cameras are not critical to the embodiments, and the cameras can vary as to frame rate, image resolution (e.g., pixels per image), color or intensity resolution (e.g., number of bits of intensity data per pixel), focal length of lenses, depth of field, etc. In general, for a particular application, any camera capable of focusing on objects within a spatial volume of interest can be used. For instance, to capture the motion of the hand of an otherwise stationary person, the volume of interest might be a meter on a side.

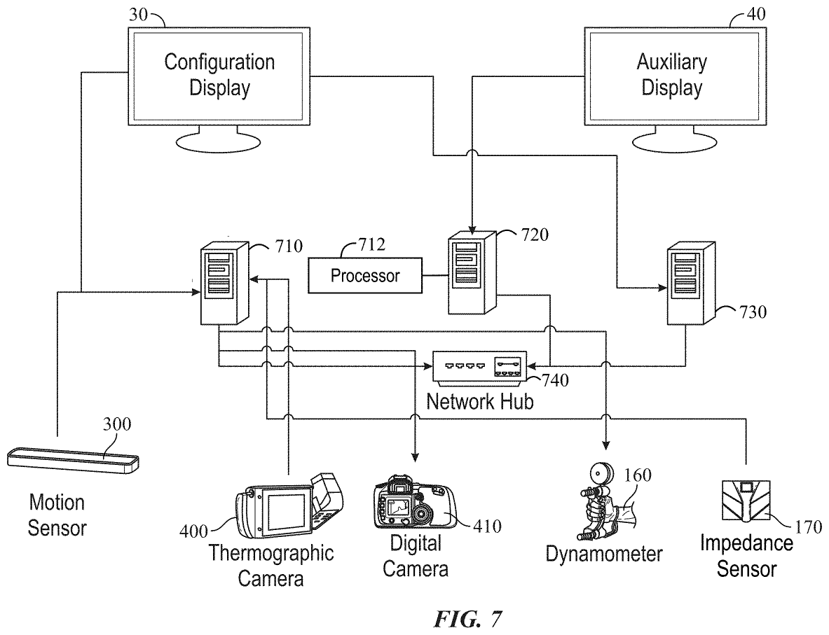

In some embodiments, the strength of the patient 105 is assessed using a dynamometer 160 (shown in FIG. 7). In the present embodiment, the dynamometer 160 is configured to assess hand and grip strength as well as key-pinch strength. In one example, two hand dynamometers 160 are utilized. Each dynamometer can be incorporated into the apparatus or presented as separate devices in communication with the apparatus and system 700.

In some embodiments, positional sensors can be used to determine which range-of-motion (ROM) stops have been engaged, and compared to what ROM limits should or should not be employed. The apparatus 10 described herein can comprise force sensors, torque sensors, and a dynamometer 160 that can be integrated to determine the strength or force/torque output of the joint and correlated to the recovery of the patient 105.

In some further embodiments, at least one of the assemblies described herein can comprise an electromyography sensor, a strain gage sensor or other sensor configured to measure strains continuously or intermittently. In some embodiments, the patient data can be used to assess motion, deflection, or provide quantifiable data of muscle growth, muscle contraction, or forces, torques or pressures resulting from a muscle contraction. The muscle contraction may be voluntary or involuntarily elicited via electrical muscle stimulation. In some embodiments, the data collected from the electromyography sensor or strain gage sensor can be utilized in a closed loop feedback control methodology to optimize/customize the electrical stimulation parameters to provide the most efficient or strongest muscle contraction for the patient. The data can also be utilized by the healthcare provider to fine tune the treatment programs based on the patient's data captured from the electromyography or strain gage sensor.

Furthermore, the apparatus 10 can be adapted and configured to engage a medical diagnostic device configured to capture data on the subject. Medical diagnostic devices typically include, for example, any device having a sensor adapted and configured to capture data from the subject (patient 105). For example, X-ray scanners, X-ray tubes with image intensifier tube, magnetic resonance scanners, infrared cameras, computed tomography scanners, ultrasound scanners, electromyography sensor units, digital camera and cameras, and electromyography sensor unit with sensors attached to the subject.

Alternative embodiments can be configured to perform pulse oximetry and can comprise a non-invasive blood pressure sensor configured to measure arterial blood pressure continuously or intermittently. In some further embodiments, a patient's heart rate can be measured in addition to sensing the patient's blood pressure. In some embodiments, one or more of the brace systems or assemblies described herein can include at least one blood pressure sensor integrated with a portion the apparatus 10. In other embodiments, the apparatus 10 can include at least one blood pressure sensor coupled to an adjacent to or some distance from the apparatus 10.

Each sensor is dimensioned to be within the housing 200 and upper surface 204 as shown in FIG. 5.

The present embodiments contemplate compatibility with all types of diagnostic imaging that are capable of producing moving images of joint motion. The method typically utilizes videofluoroscopy technology, CT scans, and magnetic resonance imaging. However, other diagnostic imaging methods such as ultrasound imaging, and imaging methods can be implemented. One skilled in the art will appreciate that as additional medical scanning or diagnostic devices become available, the present embodiments can be adapted to accommodate them.

The present embodiments contemplate the use of surface electromyography for the measurement of muscle involvement, however other diagnostic systems may be used as well in an alternative embodiment such as Mill and ultrasound or other technologies. These other diagnostic systems may or may not be sensor based. One skilled in the art will appreciate that as additional medical scanning or diagnostic devices become available, the present apparatus 10 can be adapted to accommodate them.

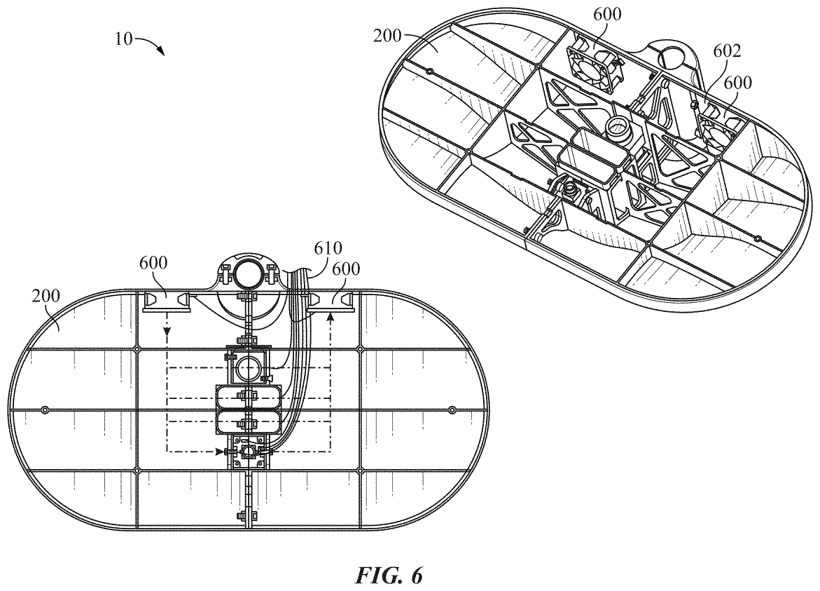

In reference to FIG. 6, a plurality of fans 600 are configured to force air from the interior of the housing 200 to the exterior to actively maintain a consistent temperature across the scanned body part 110,120. In one aspect, each fan 600 is positioned on the interior sidewall 602 of the housing 200 in fluid communication with an exhaust aperture providing a means for hot air to flow out of each assembly 110,120. In one embodiment, the plurality of fans 600 are configured to draw air into the upper and lower assemblies 110, 120 creating a cooling flow of air through the interior of the housing 200.

The component wiring 610 provides means for an electrical connection between the sensor array 25 and computer components of the apparatus 10.

In reference to FIG. 7, the configuration display 30 can provide instructions to the user as well as means for interacting with the avatar 110. Auxiliary displays 40 showing data can be used by medical professionals. It will be appreciated that the computing system is illustrative and that variations and modifications are possible. Computers 710, 720, 730 can be implemented in a variety of form factors, including server systems, desktop systems, laptop systems, tablets, smartphones or personal digital assistants, and so on. A particular implementation may include other functionality not described herein, e.g., wired and/or wireless network interfaces, media playing and/or recording capability, etc. In some embodiments, one or more cameras may be built into the computer rather than being supplied as separate components.

The computer system receives data from the sensor array 25 and stores the data in a memory component in communication with the computer system. The in memory is accessible by the healthcare professional and or the patient 105 such that patient data analysis can be performed over time. Patient data can include the patient's real-time physiological data as described hereinabove.

FIG. 8 illustrates an embodiment wherein the upper assembly 110 and lower assembly 120 are pivoted to permit the patient 105 to stand therebetween. In the illustrated embodiment, the member 106 has been rotated 90° in a counterclockwise direction such that the upper assembly 110 is pivoted to measure the feet and ankles of the patient 105. With the illustrated embodiment, the apparatus 10 can now scan the knee, lower leg, ankle, foot, and toes of the patient 105.

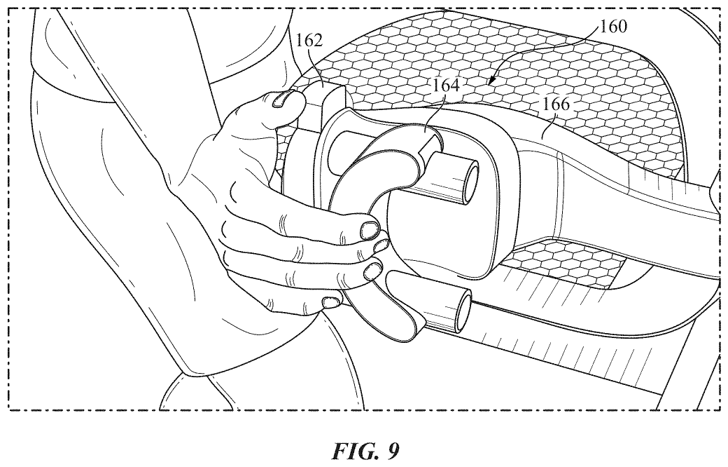

FIG. 9 illustrates a detailed view of the dynamometer 160 assembly. Bilateral supports 166 may be positioned at each side of the lower assembly 120, or suitably positioned elsewhere on the apparatus to permit the engagement with the patient's hands to assess grip and pinch strength. In some embodiments, the dynamometer includes a grip 162 and handle 164 having various sensors measuring interactions with the patient.

The sensor array 25 (which can include infrared sensors, motion sensors, dynamometers, impedance;

sensors, digital camera, and scales, in addition to any combination thereof) provides an output signal to at least one of the one or more computing devices 710, 720, 730 via network 740. The network 740 may be the Internet, a cellular network, a wired network, a wireless network, a cloud computing network, or other conventional network technology recognized in the art. It is to be understood that, in practice, there will likely be a plurality of external devices connected to the network 740. The network 740 may include a server as a unitary device but may also be implemented as a server farm or a distributed computing system to handle large capacities of data and the many simultaneous connections with computing devices 710, 720, 730. Further examples of communication networks include a local area network (“LAN”), a wide area network (“WAN”), an inter-network (e.g., the Internet), and peer-to-peer networks (e.g., ad hoc peer-to-peer networks).

The computing devices 710, 720, 730 may include conventional components such as one or more programmed applications and their interfaces, one or more memory components, and one or more processors 712. Examples of computing devices 710, 720, 730 include such known mobile devices as mobile computing devices, smartphones, desktop computers, tablets, etc., but it is to be understood that the computing devices 710, 720, 730 can be extended to other forms known in the arts.

A processor 712 suitable for the execution of a computer program includes, by way of example, both general and special purpose microprocessors and any one or more processors of any digital computing device. Generally, the processors 712 will receive instructions and data from a read-only memory or a random-access memory or both. The essential elements of a computing device are a processor 712 for performing actions in accordance with instructions and one or more memory devices for storing instructions and data. Generally, a computing device will also include, or be operatively coupled to receive data from or transfer data to, or both, one or more mass storage devices for storing data, e.g., magnetic, magneto-optical disks, or optical disks. However, a computing device need not have such devices. Moreover, a computing device can be embedded in another device, e.g., a mobile telephone, a personal digital assistant (PDA), a mobile audio or video player, a game console, or a portable storage device (e.g., a universal serial bus (USB) flash drive), to name just a few. Memory devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor 712 and the memory can be supplemented by, or incorporated in, special purpose logic circuitry.

Some embodiments described may be implemented in digital electronic circuitry, or in computer software, firmware, or hardware, including the structures, disclosed in this specification and their structural equivalents, or in combinations of one or more of them. Embodiments may be implemented as one or more computer program products, i.e., one or more modules of computer program instructions encoded on a computer readable medium for execution by, or to control the operation of, data processing apparatus. The computer readable medium may be a machine-readable storage device, a machine-readable storage substrate, a memory device, a composition of matter affecting a machine-readable propagated signal, or a combination of one or more of them. The term “processor” encompasses all apparatus, devices, and machines for processing data, including by way of example a programmable processor, a computer, or multiple processors or computers. The system 900 may include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) may be written in any form of programming language, including compiled or interpreted languages, and it may be deployed in any form, including as a standalone program or as a module, component, subroutine, or other units suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program may be deployed to be executed on one computer or on multiple computers that are located at one site or distributed across multiple sites and interconnected by a communication network. Computer program code for carrying out operations of the present embodiments may be written in an Object-oriented programming language (e.g., Java, C++, etc.). Certain components may integrate C++ dynamically linked libraries (DLL's). A .Net environment may be used for integration. The computer program code, however, for carrying out operations of the present embodiments may also be written in conventional procedural programming languages, such as the “C” programming language or in a visually oriented programming environment. Some embodiments may utilize Unity 3D and the C # programming languages.

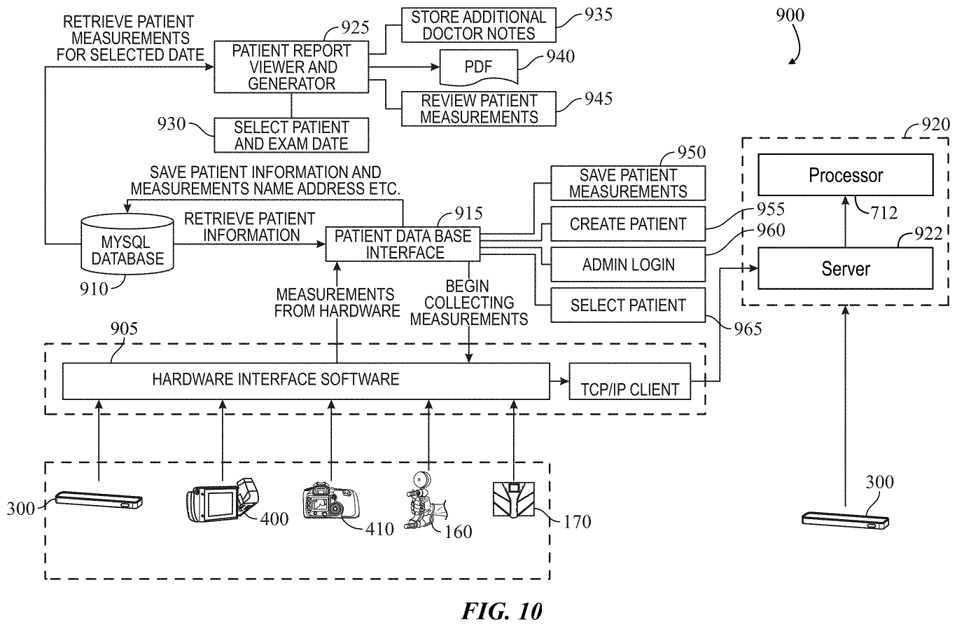

FIG. 10 illustrates the patient assessment system 900 architecture in on exemplary embodiment. Each sensor in the sensor array 25 (which can include infrared sensors, motion sensors, dynamometers, digital camera, and scales, in addition to any combination thereof) provides an output signal hardware interface software 905 which transmits patient data (measurement, etc.) from the sensor array 25 to the patient database interface 915. The memory 910 stores patient data including measurements transmitted by the sensor array 25 from current and previously stored events.

During a patient visit, the medical practitioner or patient 105 can retrieve patient data aggregated from previous events. In one example, the patient 105 has interacted with the system 900 each of the previous 12-months and stored patient data from each interaction event. The patient 105 or practitioner can select a date or set of dates to interact with a patient report viewer and patient report generator 925. The patient 105 or practitioner may select the patient exam date 930 or view practitioner notes 935, documents 940, patient data (patient measurements) and data. 945 can be reviewed, generated, stored, or interacted with. Any new patient data or amended patient data is then stored in the memory 910. Any patient data or interaction with the system 900 may be provided on interface 920 via server 922.

Before, or during an interaction with the system 900, a patient 105 or medical practitioner can save patient data 950, create a patient profile 955, login 960, select a patient 965.

The term “patient data” may refer to, but is limited to names, address, contact information, usernames, passwords, identification numbers, healthcare practitioner notes, patient notes; and any measurements transmitted by the sensors.

To provide for interaction with a user, some embodiments may be implemented on a computer having a display device e.g., an LCD (liquid crystal display) monitor, for displaying information to the user and various input/out I/O devices such as a keyboard and a pointing device, e.g., a mouse or a trackball; by Which the user may provide input to the computer. Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, or tactile input.

The operating system may be a LINUX-based operating system such as a mobile device platform; APPLE MAC OS X; MICROSOFT WIDOWS NT/WINDOWS 2000/WINDOWS XP/WINDOWS MOBILE/WINDOWS 7/8/10; a variety of UNIX-flavored operating systems; or a proprietary operating system for computers or embedded systems. The application development platform or framework for the operating system may be: BINARY RUNTIME ENVIRONMENT FOR WIRELESS (BREW); JAVA Platform, Micro Edition (JAVA ME) or JAVA 2 Platform, Micro Edition (J2ME) using the SUN MICROSYSTEMS JAVASCRIPT programming language; PYTHON™, FLASH LATE, or MICROSOFT .NET Compact, or another appropriate environment.

The device stores computer-executable code for the operating system, and the application programs such as an email, instant messaging, a video service application, a mapping application, word processing, spreadsheet, presentation, gaming, mapping, web browsing, JAVASCRIPT engine, or other applications, or example, one implementation may allow a user to access an email application, an instant messaging application, a social networking application, a video service application, a mapping application, or an imaging editing and presentation application.

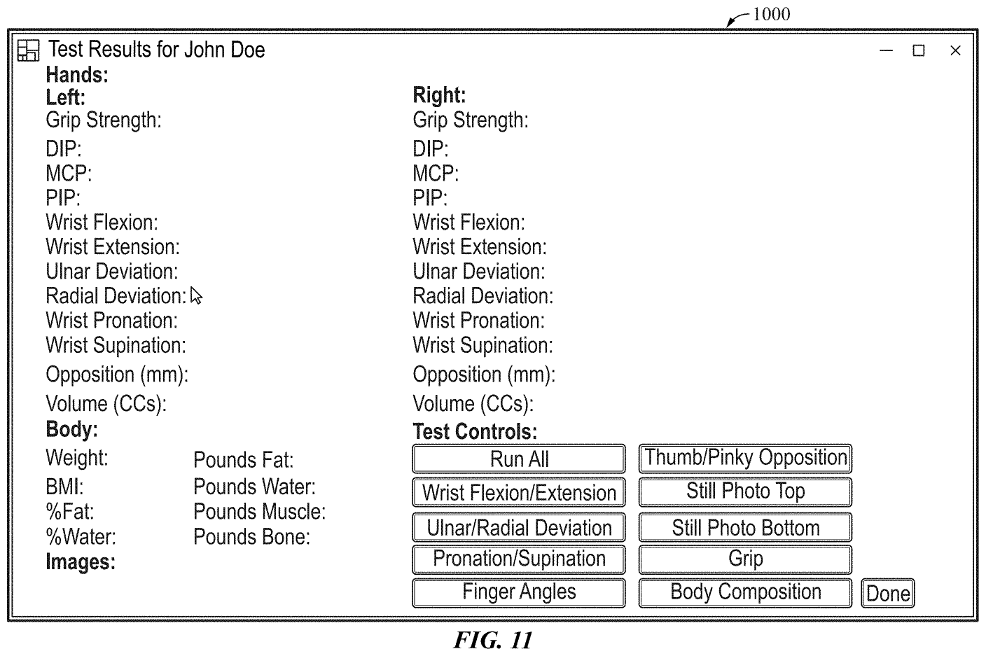

Each sensor in the sensor array 25 provides an output signal which is stored as patient data in a memory 910 (i.e., a patient database). This patient data 950 can be accessed and results viewed in a patient result interface 1000 as shown in FIG. 11. While it is understood that a patient data report can be organized in various configurations, one such configuration can compare patient data from the patient's left hand and right hand.

FIG. 11 illustrates the patient control interface 1000 having a plurality of test controls including but not limited to wrist flexion/extension, ulnar/radial deviation, pronation/supination, finger angles, thumb/pinky opposition, still photo top, still photo bottom, grip, and body composition in addition to the ability to cycle through all tests.

Each test is provided with a routine guided by an avatar 111 (shown in FIG. 1). The patient follows the routine provided by the avatar while the sensor array measure angles and movements of the patient. Each measurement is transmitted to an input field on the patient control interface 1000 or similar interface within the system.

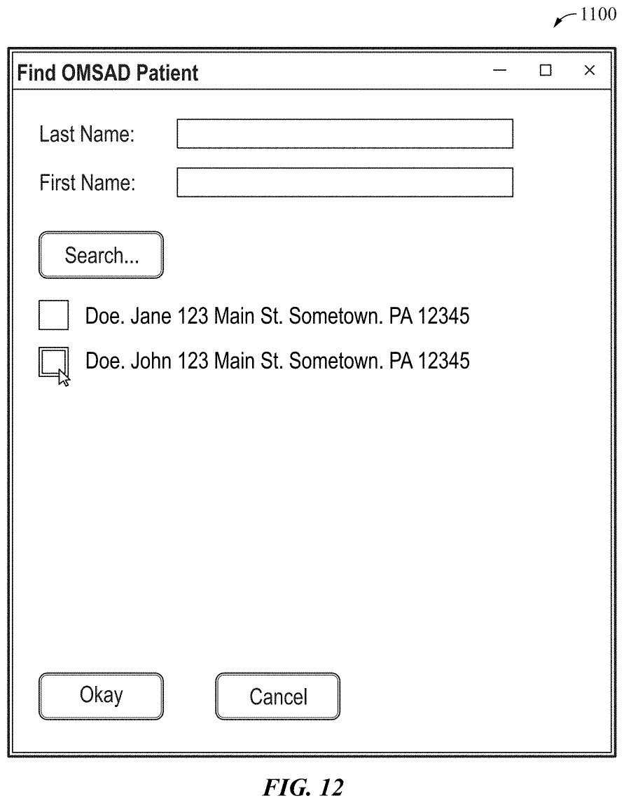

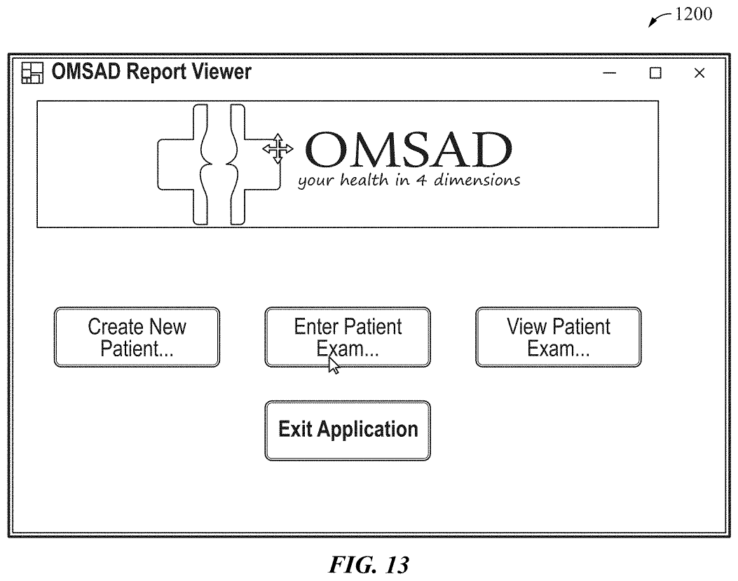

Before the patient assessment apparatus 10 measuring patient data 950 and transmitting the data to the patient assessment system 900, the patient profile 955 is either searched in the memory 910 (as shown in the patient search interface 1100 in FIG. 12), or a new patient profile 955 is created (see FIG. 13 and interface 1200). In reference to FIG. 12, if the patient is known and has already provided patient data 950 to the system 900, the user (e.g., the patient 105 or the healthcare practitioner) can search using the patient's information that has been previously provided and in communication with the select patient 965 information as shown in FIG. 10. If no previous patient 105 is found to be stored in memory 910, the system 900 provides an interface 1200 on a device display to create a new patient profile 955, enter a patient exam, or view a patient exam as illustrated in FIG. 13.

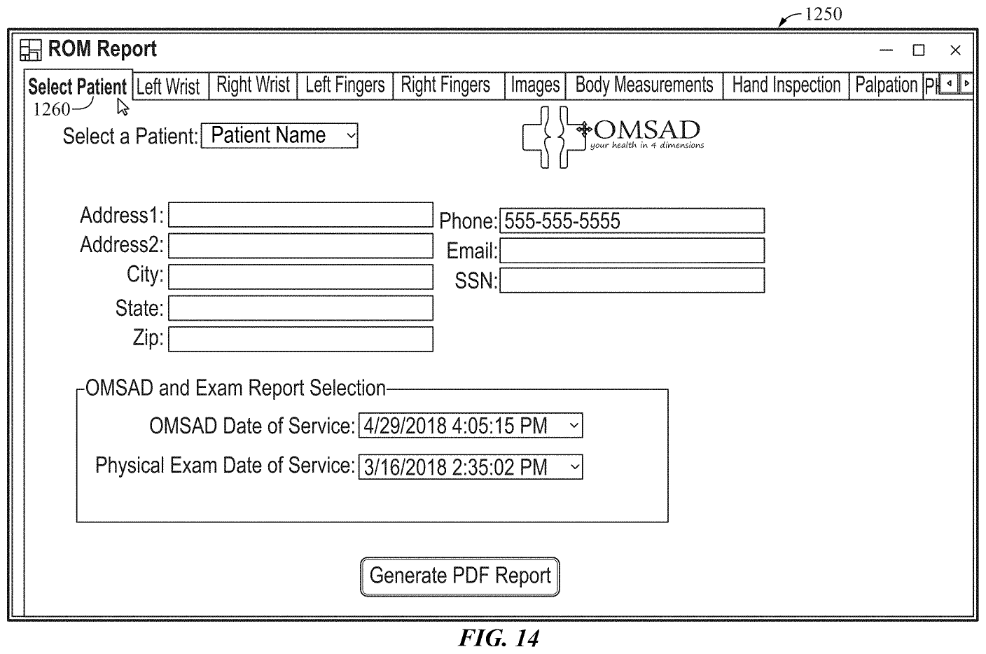

Selecting the option to enter a patient exam in FIG. 13 on a device display provides the user with report interface 1250 as illustrated in FIGS. 14-16. In FIG. 14, the user has chosen the select patient 1260 tab providing input fields for patient information, as well as a menu for selecting stored patient profiles in the memory 910. Further provided are menus for selecting or inputting an exam date and a report generation feature.

Once the patient's 105 identifying information is input into the report interface 1200, and/or 1250 the measurement process can begin. System 900 and the processor 712 thereof includes instructions to generate a network message via the server 922 which is running on an avatar 111 and is configured before the patient interaction. The server 922 receives a message from the network 740 indicating at least one routine from a list of stored routines. The processor 712 processes the message, and the indicated routine is started. If multiple routines are indicated, each routine will be provided by the avatar 111 in a pre-programmed order. Routines may be added, removed, or rearranged depending on the needs of the particular patient whom which the avatar 111 is interacting.

Following the execution of the one or more routines, the patient data 950 is stored in the memory 910 along with the patient identifier, a date, and a time to indicate when the patient data was acquired. The patient data can be calculated and tabulated in a shareable format such that the exam information and patient data can be shared with various healthcare providers or other persons having access to the patient portal within the system 900.

While routines which collect patient data 950 may vary, an example is provided for the hand and wrist measurements of a patient. Initially, the system 900 provides at least one interface to interact and direct the patient 105. The system 900 further provides at least one interface for data control, manipulation, and analysis by medical practitioners. Next, the system collects optical images using one or more cameras 410 or similar optical sensors. Data can be collected and provided by the dynamometer 160 and impedance sensor 170. In some embodiments, 4-point in-body analysis using electrical impedance measured by an impedance sensor 170, determines body fat measurements of the patient 105. Patient data 950 is then stored in the memory 910.

The impedance sensor 170 operates to measure body composition. For example, lean body mass for particular anatomy (such as each arm) can be measured and compared. The impedance sensor 170 may be used to elucidate muscle wasting in a particular area. The impedance tests may be performed using bio-electrical impedance analysis (BIA).

A set of routines are provided by the avatar 111 to measure flexion, extension, radial and ulnar deviation, supination, abduction, adduction, and opposition of the fingers, thumb, palm, and wrist. Next, the avatar 111 provides a routine for ulnar deviation, abduction, adduction, and opposition. Further, a routine is provided for supination and pronation of the hand and wrist.

The avatar 111 also provides routines for the foot and ankle. Referring to the ankle, routines can include ankle plantar and dorsiflexion, ankle supination, and ankle pronation. Referring to the foot, toe extension, toe flexion, and resting position can be measured.

In some embodiments, optical imagery is compiled for the hand and wrist in 3-dimensions for each routine provided by the avatar 111 while the patient performs the routine. The system 900 can compare patient data sets from each routine and measurement taken therefrom. Next, volumetric data and flexibility measurements are stored for the hands and wrists.

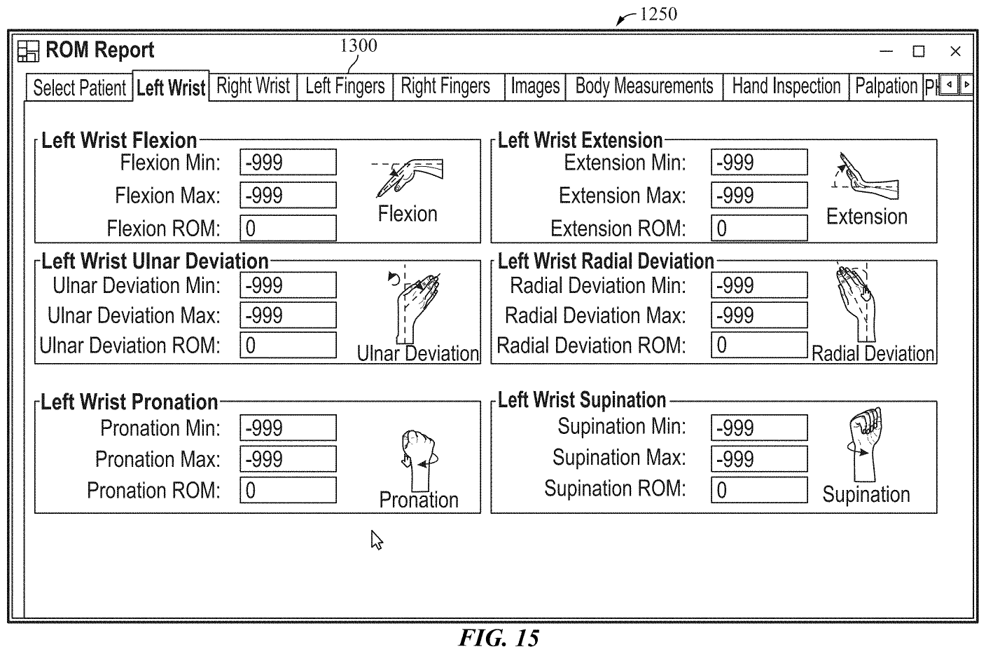

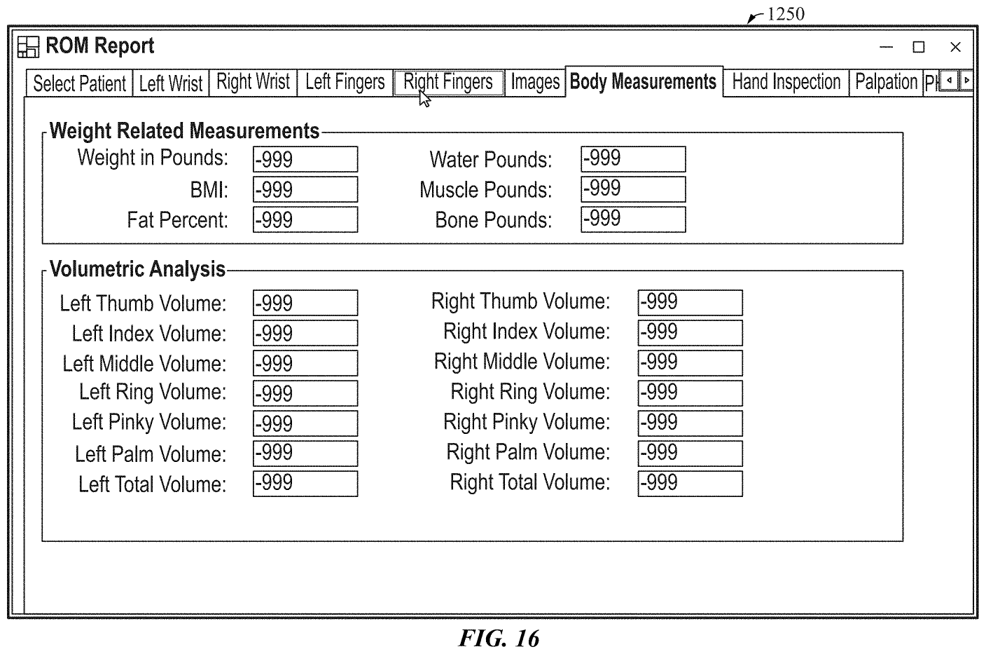

The report interface 1250 provides a plurality of selectable tabs 1300 which organize the patient data 950 by target anatomy, data type, or similar useful means for organizing physiological data. For example, selectable tabs 1300 can include individual tabs not limited to the left wrist, right wrist, left fingers, right fingers, images, body measurements, hand inspection, palpation, etc. FIG. 15 illustrates the report interface 1250, and more specifically, the left wrist report (selected from the selectable tabs 1300). Patient data 950 received from the sensor array 25 and input fields thereof can include values for left wrist flexion, left wrist extension, left wrist ulnar deviation, left wrist radial deviation, left wrist pronation, and left wrist supination. In some embodiments related to movement, data can include minimum and maximum data points calculated from a range-of-motion total. FIG. 16 illustrates the report interface 1250, and more specifically, the body measurement report (selected from the selectable tabs 1300). Patient data 950 and the input fields thereof can include weight-related measurements (e.g., weight, BMI, fat percentage, water weight, muscle weight, bone weight, and other useful metrics known in the arts), and volumetric analysis (e.g., volumes for each finger, palm, and total volume).

To generate and present any particular patient data 950 as shown and described herein system 900 may have an interface module (in communication with the displays), a collection module (in communication with the sensor array 25), a transformation module (in operable communication with the processor 712), a presentation and/or comparison module (in operable communication with the processor 712, and/or a storage module, in operable communication with the memory 910.

The interface module may be configured to obtain or receive raw patient data from any sensor in the sensor array 25, either directly from the sensor array 25, or from the patient 105 and attending physicians or nurses. The collection module may collect the raw medical data from interface module, and further may collect additional material from storage module in operable communication with the memory 910, including the patient's historical medical data sets as well as other required general medical data (optional statistics). In some embodiments, the raw medical data may be transmitted to the transformation module, and the stored and historical medical data may be sent to presentation and/or comparison module. In some embodiments, the medical and historical data may be sent to the transformation module and/or the presentation and/or comparison module.

The transformation module may receive incoming raw medical data and may convert this data into a usable format for generating the patient data. The transformed data may then be sent to the combination module, which in turn may generate the patient data 950, using a predetermined calculation method.

A comparison module may receive the calculated patient data and may prepare a plurality of reports as shown as described herein. A storage module may be configured to store and retrieve patient data at various times.

Storage of patient data 950 in the memory 910 provides the ability for data to be stored and compared over time for each patient, or across populations. Those skilled in the arts will find many uses for comparing patient data 950 over time. In one example, a particular patient 105 has interacted with the patient assessment apparatus 10 and system 900 over a 300-day period of time, which has consisted of a first scan at day 1, and a second scan at day 300. Comparing the patient's data 950 over the 300-day period shows an interval decrease in gait speed, cadence, step length, stride length, and single limb support time. Meanwhile, the patient's data 950 shows an interval increase in step and stride time, double limb support time, and step width. Further, a 1.5 cm contraction (from 4.5 cm to 3 cm) of the width of a scar on the left hand has been measured while a +10° interval increase in thenar eminence temperature has been shown on the right hand. A volumetric analysis has shown a decrease from 70 mm3 to 30 mm3 over the 300 day time period. Hand and wrist motion have not differed significantly, and the left-hand grip strength has decreased from 120 pounds to 85 pounds. Meanwhile, pinch strength has remained stable.

The above example can be used, potentially in view of other observations or measurements, to indicate or infer a variety of physiological occurrences.

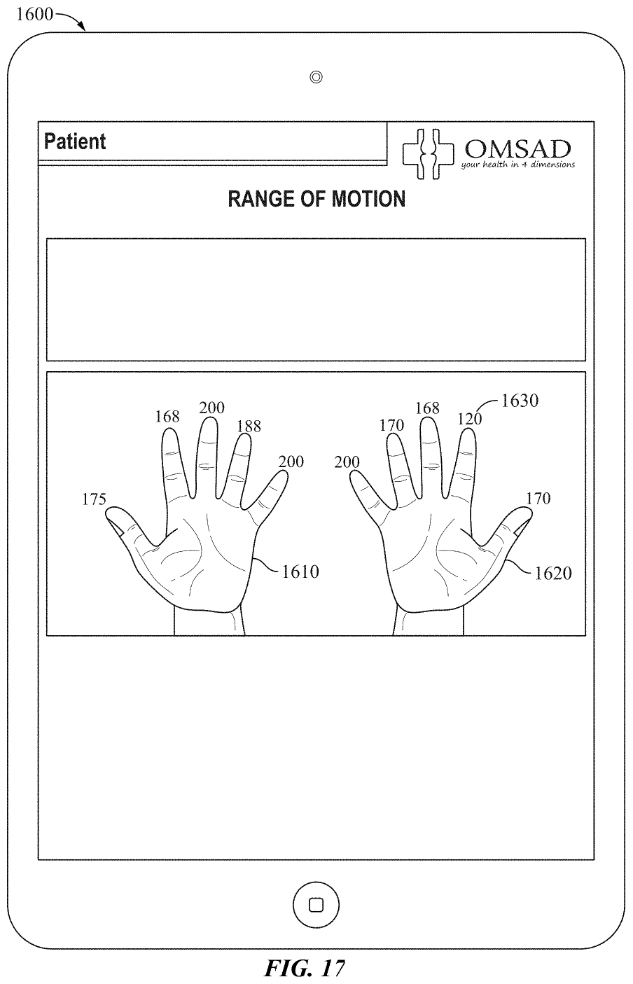

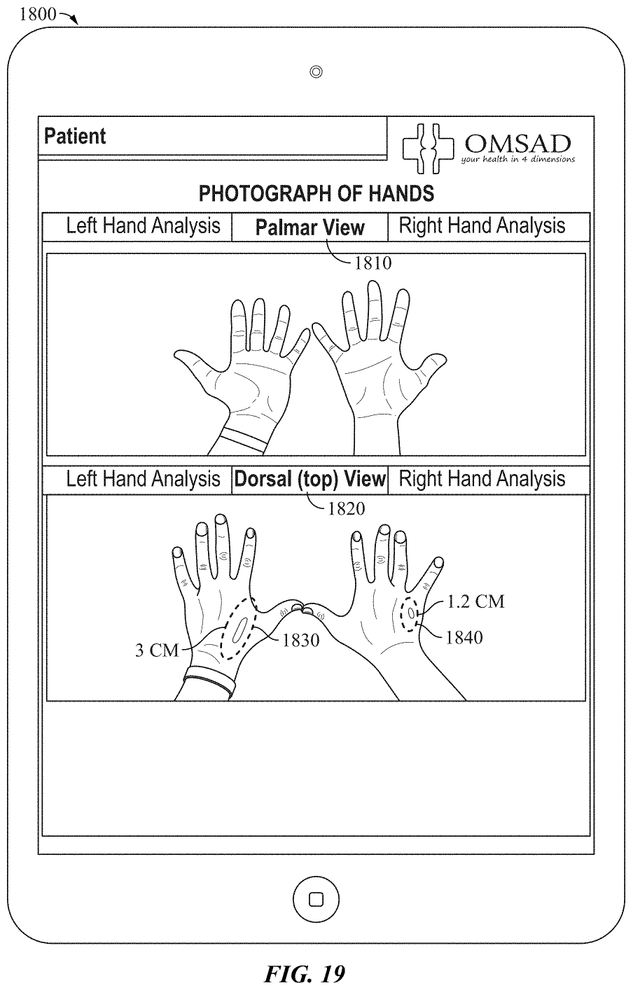

In some embodiments, the patient report and review generator 925 provides output reports on a device display to both the patient 105 and the medical practitioner. FIGS. 17-21 show examples of various report interfaces generated following the transmission and processing of patient data 950. Patient data 950 can be manually entered, or automated based on predetermined protocols. These interfaces may be viewed as a modular interface, or as a fixed report (such as a .pdf documents 940). Further, each report and additional embodiments thereof can be stored in the memory 910 for future use. FIG. 17 shows a range-of-motion (ROM) report interface 1600 showing the right hand 1610 and left hand 1620 of the patient 105 with each digit having range-of-motion values 1630. Each value 1630 can be used in various ways to determine range-of-motion of the target anatomy (such as each digit, the wrist, the arm, the ankles, toes, etc.). In some embodiments, total active motion (TAM) can be calculated for the target anatomy. In the example provided by FIG. 17, TAM can be calculated by adding the ROM of each joint for each digit. The sum of the extension for the metacarpophalangeal joint (MCP), proximal interphalangeal joints (PIP), and distal interphalangeal joints (DIP) is subtracted from the total achievable flexion of the same joints.

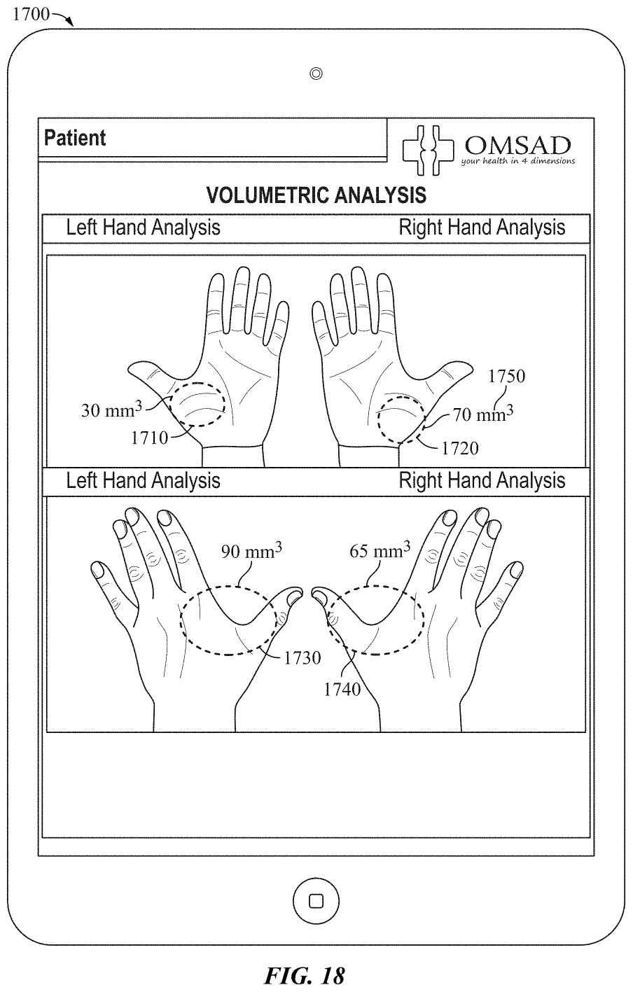

FIG. 18 illustrates a volumetric analysis interface 1700 having the dorsal and palmar views of the right and left hands. In one example, the left and right-hand palmar target anatomies 1710, 1720 and right and left-hand dorsal target anatomies 1730, 1740 are volumetrically analyzed yielding a plurality of values (1750 in on such example).

Now referring to FIG. 18, the photographic report interface 1800 is shown having palmar and dorsal views for each of the left and right hands of the patient. While shown in FIG. 18, each interface shown in any of FIGS. 17-21 may be provided with selection tabs 1810, 1820 to toggle between views or target anatomies of the patient. The photographic report interface 1800 may provide patient data 950 related to measurements of the target anatomy as illustrated in measurements 1830, 1840. Other visual data can be collected such as skin coloration, and various other metrics known in the arts.

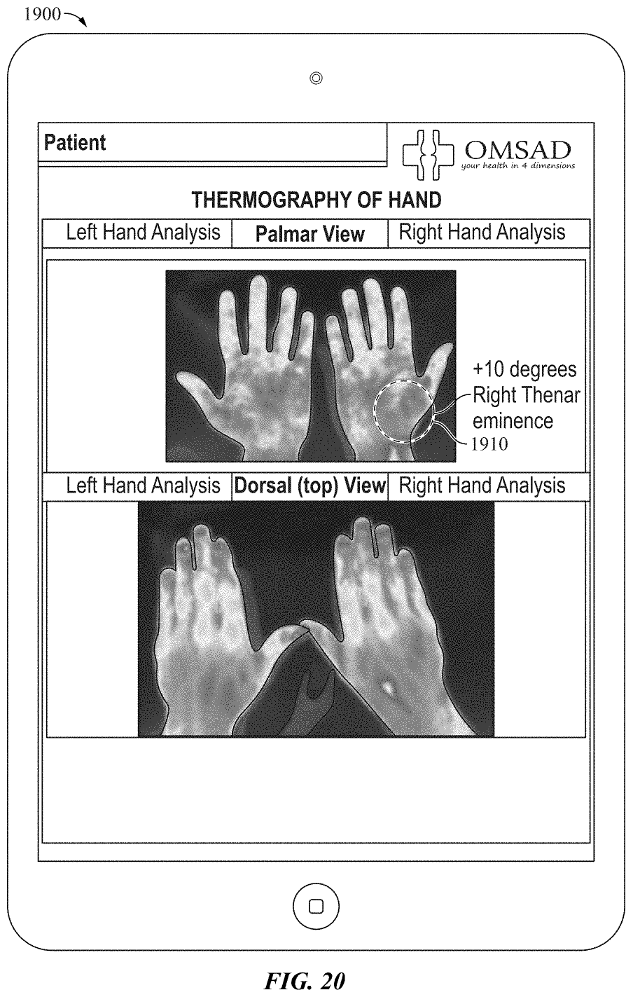

FIG. 20 illustrates a thermographic report interface 1900 having the palmar and dorsal views for each of the right and left hands of the patient 105. Thermographic data 1910 such as temperature and other useful measurements gained from infrared sensors can be provided.

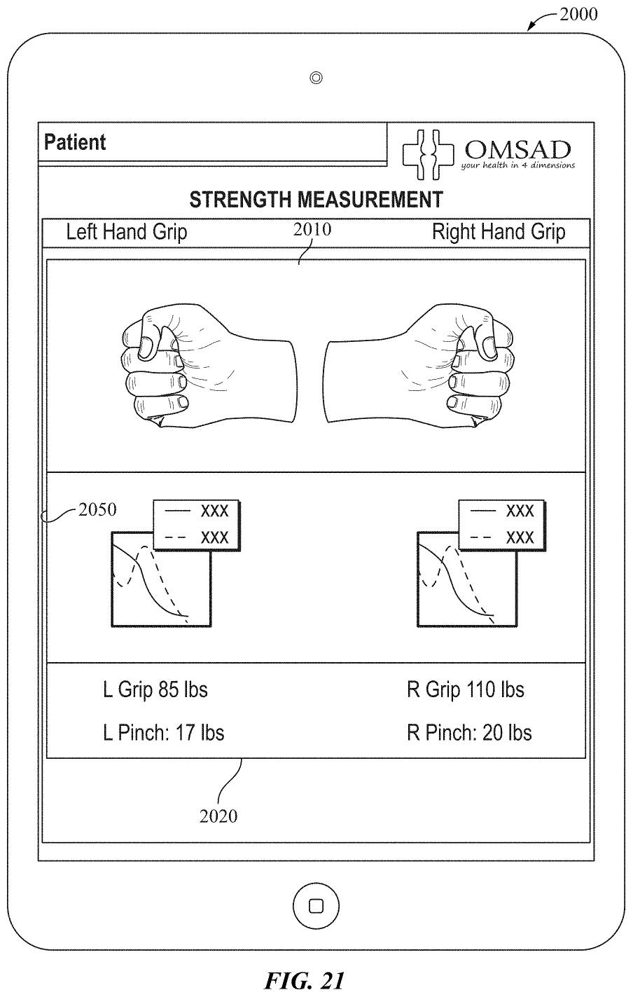

FIG. 21 shows a strength measurement interface 2000 which can include grip imagery 2010 and strength output measurements 2050, 2070 for each hand of the patient 105.

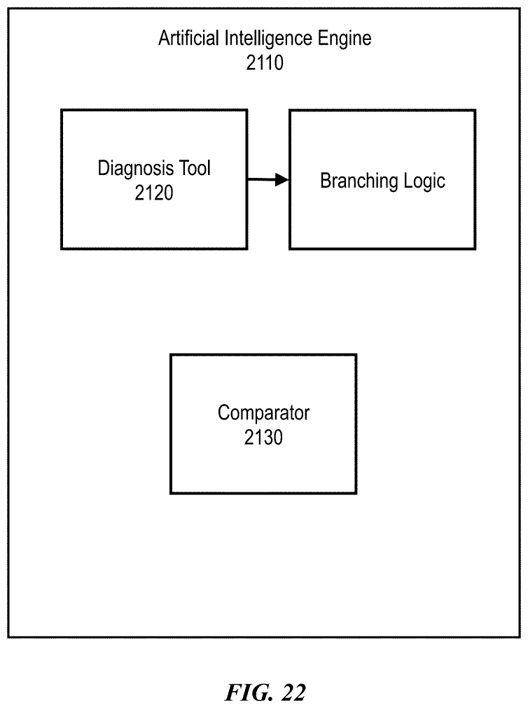

Now referring to FIG. 22, once the patient 105 performs the routine, each measurement from the sensor array 125 is stored in the memory 910. Data can be analyzed by an artificial intelligence engine 2110 to interpret each data point, compare data points to historical data, and determine potential diagnosis if a threshold value of factors is reached. For example, a diagnosis tool 2120 can receive volumetric data, optical data, and impedance data and compare the data to historical or control data using a comparator 2130 to point towards a potential diagnosis.

In some embodiments, the sensor array 125 is in operable communication with system for guiding physical positioning, orientation, and motion of the human body, comprising a cloud computing-based subsystem including an artificial neural network and spatial position analyzer.

The diagnosis tool 2120 is a diagnostic tool for detecting and differentiating orthopedic abnormalities associated with various conditions or diseases. Such diagnosis is based on the patient's data retrieved from sensor array 125, with or without other clinical data from which may be manually input by the medical professional. Various technology tools are used to detect and highlight abnormalities in the patient's anatomy. Examples of such technology tools include branching logic, such as branching logic used with the history of present illness (HPI), artificial intelligence algorithms such as artificial neural networks for computer vision, machine learning, and statistical pattern recognition and digital image processing. These technologies are employed to extract the characteristics and features of the anatomy associated with a variety of conditions and diseases for diagnosis purposes.

Many characteristics of the patient's anatomy may be analyzed for diagnostic purposes, such as quantitative measurements, texture descriptors, anatomical spatial descriptors, and other specific information descriptors. Quantitative measurements are a set of quantifiable features that can be used to assess the existence or degree of an abnormality, such as size, shape, intensity and various statistics of such measurements. The texture descriptors characterize the homogeneity of an area that can be used as diagnostic indicators, for example, tissue degeneration. The anatomical spatial descriptors can be used to indicate precise, relative positions of structures of the anatomy. For example, necrosis treatment varies with location, and the size and location of the necrotic lesion are important factors for elucidating a diagnosis for particular disease.

In another aspect, some embodiments may comprise a computational framework, artificial neural network, and application instruction set operable on general-purpose devices.

In some embodiments, the avatar functions as a system for guiding physical positioning, orientation, and motion of the human body, comprising a cloud computing-based subsystem including an artificial neural network and spatial position analyzer, the cloud computing-based subsystem being adapted for data storage, management and analysis. The artificial intelligence engine may transfer the report data and analyzed data to the report generator.

In some embodiment, the artificial intelligence engine intakes data received during a routine performed by the patient. The artificial intelligence engine may alter the routine depending on the received patient data values.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A patient assessment system comprising:
a computing system including a processor configured to provide a plurality of routines to a patient using an avatar via a display;
a sensor array configured to capture patient data and transmit patient data to a memory in operable communication with the processor, the memory configured to store a plurality of patient data received from the sensor array;
an artificial intelligence engine in operable communication with the memory, the artificial intelligence system configured to analyze patient data one or more abnormalities; and
a patient report generator in operable communication with the artificial intelligence engine, the patient report generator configured to provide a plurality of reports to an interface configured to display the plurality of reports.

2. The system of claim 1, wherein the sensor array includes at least one of the following:
at least one infrared sensor;
at least one photographic device; and
at least one dynamometer.
at least one impedance sensor

3. The system of claim 2, wherein the sensor array is configured to transmit at least;
volumetric data;
thermographic data;
body composition data;
strength data; and
optical imaging data,
wherein the volumetric data, the thermographic data, the body composition data, the strength data, and the optical image data is aggregated into at least one data set.

4. The system of claim 3, wherein the artificial intelligence engine is comprised of:
a comparator configured to compare at least one data set stored in the memory to determine a difference between two or more values in the at least one data set;
a diagnosis tool to determine if the difference is above a threshold value, and wherein the threshold value is used to elucidate a diagnosis and transmit the diagnosis to at least one of the plurality of reports.

5. The system of claim 4, wherein the plurality of reports is stored in the memory, and wherein a comparing module compares the plurality of reports for each patient over time.

6. The system of claim 5, wherein the user selects at least one of the plurality of routines, wherein the sensor array transmits the patient data to the artificial intelligence engine, wherein the artificial intelligence engine changes the routine in view of the patient data.

7. A patient assessment system comprising:
a sensor array configured to provide a plurality of patient data to a computing system, the computing system including at least one processor operably coupled to the sensor array, the at least one processor configured to perform the following:
providing a plurality of routines to a patient;
collecting a plurality of patient data provided by the sensor array;
transmitting the plurality of patient data to an artificial intelligence engine;
analyzing, via the artificial intelligence engine, the patient data to detect a potential abnormality; and
generating a plurality of reports using the patient data,
a memory operably coupled to the at least one processor and configured to store the plurality of patient data; and
a server operably coupled to the memory and configured to receive a request for the plurality of patient data, and to serve the plurality of patient data in response to the request.

8. The system of claim 7, wherein the plurality of routines are stored in the memory, wherein the server receives a request for at least one routine and serves the at least one routine in response to the request, wherein the at least one routine is provided to the patent via a display.

9. The system of claim 8, wherein the plurality of routines is performed by an avatar on a display.

10. The system of claim 9, including the avatar receiving instructions from the at least one processor to provide the plurality of routines to the patient, wherein the patient and the avatar simultaneously perform the plurality of routines provided on the display.

11. The system of claim 7, wherein the patient performs the at least one routine, the processor is configured to measure the plurality of patient data received from the sensor array, wherein the plurality of patient data is related to at least one target anatomy of the patient and stored for comparison in the memory.

12. The system of claim 11, wherein the artificial intelligence engine is comprised of:
a comparator configured to compare at least one data set stored in the memory to determine a difference between two or more values in the at least one data set;
a diagnosis tool to determine if the difference is above a threshold value, and wherein the threshold value is used to elucidate a diagnosis and transmit the diagnosis to at least one of the plurality of reports.

13. The system of claim 12, wherein the plurality of reports is stored in the memory, and wherein a comparing module compares the plurality of reports for each patient over time.

14. The system of claim 11, wherein the target anatomy of the patient includes at least one of the following: wrists, hands, forearms, fingers, ankles, toes, and feet.

15. The system of claim 7, wherein the sensor array includes at least one of the following:
at least one infrared sensor configured to collect and transmit range-of-motion data
at least one infrared sensor configured to collect and transmit thermographic and calculate volumetric data;
at least one impedance sensor, configured to collect biometric data;
at least one photographic device, configured to collect optical imaging data; and
at least one dynamometer, configured to collect strength data.

16. A patient assessment system comprising:
a sensor array configured to provide a plurality of patient data to a computing system, the computing system including at least one processor operably coupled to the sensor array, the at least one processor configured to perform the following:
providing, via an avatar, a plurality of routines to a patient, each of the plurality of routines adapted to aggregate data into a plurality of data sets;
transmitting the aggregated data to a patient interface adapted to permit user interaction therewith;
transmitting the plurality of patient data to an artificial intelligence engine;
analyzing, via the artificial intelligence engine, the patient data to detect recovery progress or a potential abnormality; and
generating a plurality of reports using the patient data,
a memory operably coupled to the at least one processor and configured to store the plurality of patient data in a shareable document format; and
a server operably coupled to the memory and configured to receive a request for the plurality of patient data and to serve the plurality of patient data in response to the request.

17. The system of claim 16, wherein the plurality of sensors includes at least one of the following:
at least one infrared sensor configured to collect and transmit range-of-motion data, thermographic data, and volumetric data;
at least one impedance sensor to collect impedance data;
at least one photographic device configured to provide imaging data; and
at least one dynamometer, configured to provide strength data.

18. The system of claim 17, wherein the artificial intelligence engine is comprised of:
a comparator configured to compare at least one data set stored in the memory to determine a difference between two or more values in the at least one data set;
a diagnosis tool to determine if the difference is above a threshold value, and wherein the threshold value is used to elucidate a diagnosis and transmit the diagnosis to at least one of the plurality of reports.

19. The system of claim 18, wherein the plurality of reports is stored in the memory, and wherein a comparing module compares the plurality of reports for each patient over time.

20. The system of claim 19, wherein the plurality of reports are compared, via the comparator, to a standardized data sets stored in the database.
